(19) United States
(12) Patent Application Publication
(10) Pub. No.: US 2010/0076521 A1
(43) Pub. Date: Mar. 25, 2010

(54) Electrical stimulation system and method for generating virtual channels

(75) Inventors: Charles Tak Ming Choi, Hsinchu City (TW); Chen Hua Hsu, Jhubei City (TW); Yeng Ting Lee, Taoyuan City (TW)

Correspondence Address: BACON & THOMAS, PLLC, 625 SLATERS LANE, FOURTH FLOOR, ALEXANDRIA, VA, 22314-1176, US

(73) Assignee: National Chiao Tung University, Hsinchu City (TW)

(21) Appl. No.: 12/461,944

(22) Filed: Aug. 28, 2009

(30) Foreign Application Priority Data
Sep. 25, 2008 (TW) 097136818

(51) Int. Cl. A61N 1/08

(52) U.S. Cl. 607/59

(57) ABSTRACT

Electrical stimulation system and method for generating virtual channels are disclosed. The electrical stimulation system comprises: an electrode controller, a carrier, a plurality of electrode units, and a buffer layer. The electrode units are disposed on the carrier, and each of the electrode units are electrically connected to the electrode controller independently. Besides, the electrode units and the carrier are covered with the buffer layer. When the electrode controller receive a control signal and drive the corresponding electrode units, the electrical currents output from the corresponding electrode units can electrically interfere with each other to generate a virtual channel between the corresponding electrode units.

### BACKGROUND OF THE INVENTION

1. Field of the Invention

The present invention relates to an electrical stimulation system for generating virtual channels and, more particularly, to an electrical stimulation system for generating virtual channels which can be applied to implants or medical devices to improve the resolution of neural stimulation.

2. Description of Related Art

It is known that organic pathological changes or organ degeneration may occur with the elevated age of a patient, or organ damage may be caused by external force. Especially, the loss of sight or hearing may affect patients' lives greatly. The main reason for loss of sight or hearing is that the functions of the receptors do not work properly, which are used to receive external signals, such as light waves, or sound waves. Hence, it is impossible to transfer the external signals into electrical stimulation to stimulate neural fibers. Therefore, the external signals cannot be transmitted to the patients' brains, which may cause the patients' loss of their sight or hearing.

In recent years, retinal prosthesis and cochlear implants have been developed to restore patients' vision or hearing. The retinal prosthesis is an electrode array, which is applied to replace the photoreceptor cells to receive signals and stimulate optic nerve fibers. Hence, the retinal prosthesis can be used for patients' with retinal diseases. The patient with retinal prosthesis has to wear glasses fitted with a micro-camera to receive light waves. Then, the light waves are transferred into signals, which can be accepted by eyes. After the electrode array receives the signals, the signals are transferred into currents to stimulate nerve fibers. Finally, the signals can be transmitted to the patient's brain through the nerve fibers.

In addition, Parkinson's disease is a degenerative disease that usually occurs in elderly patients. The primary symptom is caused by the insufficient formation and action of dopamine, which is produced in the dopaminergic neurons of the brain. When more than 50% of the dopaminergic neurons have degenerated, the patients may lose their motion control. Currently, in addition to drug therapy, there are several surgical treatments for Parkinson's disease, i.e. lesion procedure, deep brain stimulation, and restorative therapy. Deep brain stimulation (DBS) is a surgical treatment involving the implantation of a medical device called an electrical stimulation lead, which comprises an electrode array. The electrical impulses generated from the electrode array are sent to specific parts of the brain to inhibit the signals causing the abnormal motion. Hence, it is possible to treat the disorder in motion by deep brain stimulation.

The aforementioned retinal prosthesis and electrical stimulation lead of deep brain stimulation are one kind of electrical stimulation system, which comprises of an electrode array installed around the target neural fibers. In the conventional electrical stimulation system, the current generated from an electrode unit of the electrode array can only stimulate the neural fiber, which corresponds to the position of the electrode unit. Hence, it is impossible to stimulate the neural fibers that are positioned in a region between two adjacent electrode units selectively.

Therefore, it is desirable to provide an electrical stimulation system, which can improve the resolution of neural stimulation. Thus, the electrode units can not only stimulate the neural fibers corresponding to the positions of the electrode units, but also stimulate the neural fibers located in a region between two adjacent electrode units selectively.

### SUMMARY OF THE INVENTION

The object of the present invention is to provide an electrical stimulation system for generating virtual channels, which can form a virtual channel between electrodes and nerve fibers to improve the resolution of neural stimulation. Also, the electrical stimulation system for generating virtual channels of the present invention can be applied to implants or medical devices.

Another object of the present invention is to provide an electrical stimulation method for generating virtual channels, which can improve the resolution of neural stimulation to achieve the effect of stimulating nerve fibers accurately.

To achieve the aforementioned objects, the present invention provides an electrical stimulation system for generating virtual channels, which comprises: an electrode controller, a carrier, a plurality of electrode units, and a buffer layer. In the electrical stimulation system for generating virtual channels of the present invention, the plurality of electrode units is disposed on the carrier, and each of the electrode units is electrically connected to the electrode controller independently. Besides, the carrier and the electrode units are covered with the buffer layer. When the electrode controller receives a control signal to drive the corresponding electrode units, the currents output from the corresponding electrodes units electrically interfere with each other to generate a virtual channel between the corresponding electrode units.

Furthermore, the present invention provides an electrical stimulation method for generating virtual channels, which comprises the following steps: providing an electrical stimulation system, which comprises an electrode controller, a carrier, a plurality of electrode units, and a buffer layer, wherein the electrode units are disposed on the carrier, each of the electrode units are electrically connected to the electrode controller independently, and the carrier and the electrode units are covered with the buffer layer; inputting a control signal to the electrode controller of the electrical stimulation system, and driving the corresponding electrode units to output currents; generating a virtual channel between the corresponding electrode units through the currents output from the corresponding electrodes electrically interfering with each other.

The electrical stimulation system and method for generating virtual channels can prevent the direct contact between the electrode units and nerve fibers, due to the electrode units being covered with the buffer layer. Moreover, it is possible to generate a virtual channel by the electrical stimulation system and method for generating virtual channels of the present invention, as the currents output from the electrode units electrically interfere with each other in and through the buffer layer. Besides, each of the electrode units is electrically connected to the electrode source voltage or current controller independently, so it is possible to control the position of the virtual channel by means of adjusting the voltage or current ratio input into the electrode units. Hence, the electrical stimulation system and method for generating virtual channels of the present invention not only can direct stimulate the nerve fibers in the vicinity of the electrode units, but also can stimulate the nerve fibers positioned outside the the immediate vacinity of the electrode units by adjusting the stimulating sites or creating and adjusting the virtual channels. Therefore, the electrical stimulation system and method for generating virtual channels of the present invention can improve the spatial resolution of the nerve stimulation.

In the electrical stimulation system and method for generating virtual channels of the present invention, the corresponding electrode units driven by the electrode controller comprise at least two adjacent electrode units. Furthermore, the virtual channel is generated between the at least two adjacent electrode units.

Preferably, in the electrical stimulation system and method for generating virtual channels of the present invention, the electrode units of the electrical stimulation system form an m×n array, and each m, and n is independently an integer of 1 or more.

In the electrical stimulation system and method for generating virtual channels of the present invention, the electrical stimulation system can further comprise at least one anchor, which is disposed in the buffer layer and protrudes from the outer surface of the buffer layer. For example, the anchors may be installed on the carrier, penetrating through the buffer layer, and protruding from the outer surface of the buffer layer; or the anchors may be installed on the buffer layer and protrude from the outer surface of the buffer layer.

In the electrical stimulation system and method for generating virtual channels of the present invention, the conductivity of the buffer layer of the electrical stimulation system can be within a range of 0.01 to 100 simens/m. Preferably, the conductivity of the buffer layer of the electrical stimulation system is within a range of 0.1 to 10 simens/m. Besides, the thickness of the buffer layer can be within a range of 5 to 10000 μm. Preferably, the thickness of the buffer layer is within a range of 5 to 100 μm.

In the electrical stimulation system and method for generating virtual channels of the present invention, the distance between the two adjacent electrode units of the electrical stimulation system can be within a range of 10 to 10000 μm. Preferably, the distance between the two adjacent electrode units of the electrical stimulation system is within a range of 10 to 100 μm.

In the electrical stimulation system and method for generating virtual channels of the present invention, the material of the carrier of the electrical stimulation system is a biocompatible insulating material, such as silicone, polyimide, or fluoropolymer resin.

In the electrical stimulation system and method for generating virtual channels of the present invention, the material of the buffer layer of the electrical stimulation system is a biocompatible semiconducting material. Besides, the buffer layer of the electrical stimulation system can further comprise a buffer, wherein the components of the buffer are similar to the components of tissue fluid.

Therefore, the buffer layer and the carrier of the electrical stimulation system of the present invention are made of biocompatible materials, so it is possible to apply the electrical stimulation system of the present invention to implants or medical devices, such as a retinal prosthesis, a cochlear implant, the electrical stimulation lead for deep brain stimulation, or spinal cord stimulation. Furthermore, the material of the buffer layer is a semiconducting material, which still has conductivity, so the buffer layer allows the electrical stimulation to be generated therein without impeding the currents output from the electrode units greatly. In addition, the material of the buffer layer is a non-perfect electric conductor, so the situation of short circuits will not happen. Moreover, the electrical stimulation system and method of the present invention can generate a virtual channel between two adjacent electrode units. Hence, it is possible to stimulate target nerve fibers accurately by the electrical stimulation system and method of the present invention, even though the number of the electrode units is limited. Therefore, the electrical stimulation system and method of the present invention can improve the spatial resolution of the neural stimulation.

Other objects, advantages, and novel features of the invention will become more apparent from the following detailed description when taken in conjunction with the accompanying drawings.

### BRIEF DESCRIPTION OF THE DRAWINGS

FIG. 1 is a perspective view of the electrical stimulation system for generating virtual channels as a retinal prosthesis in Embodiment 1 of the present invention;

FIG. 2 is a cross-sectional view of the electrical stimulation system for generating virtual channels installed on the retinal in Embodiment 1 of the present invention;

FIG. 3 is a cross-sectional view of the electrical stimulation system installed on the retinal in Comparative example 1;

FIG. 4 is a stimulation model of the electrical stimulation system of Comparative example 1;

FIG. 5 is a stimulation model of the electrical stimulation system for generating virtual channels of Embodiment 1 of the present invention;

FIG. 6 is another stimulation model of the electrical stimulation system for generating virtual channels of Embodiment 1 of the present invention;

FIG. 7 is a cross-sectional view of the electrical stimulation system for generating virtual channels installed on the retinal in Embodiment 2 of the present invention;

FIG. 8 is a perspective view of the electrical stimulation system for generating virtual channels as a retinal prosthesis in Embodiment 3 of the present invention;

FIG. 9 is a perspective view of the electrical stimulation system for generating virtual channels used in the deep brain stimulation in Embodiment 4 of the present invention;

FIG. 10 is a perspective view of the electrical stimulation system for generating virtual channels of Embodiment 4 of the present invention;

FIG. 11 is a perspective view of the electrical stimulation system for generating virtual channels in Embodiment 5 of the present invention;

FIG. 12 is a perspective view of the electrical stimulation system for generating virtual channels used in the spinal cord stimulation in Embodiment 5 of the present invention; and

FIG. 13 is a cross-sectional view of the electrical stimulation system for generating virtual channels used in the spinal cord stimulation in Embodiment 5 of the present invention.

### DETAILED DESCRIPTION OF THE PREFERRED EMBODIMENT

### Embodiment 1

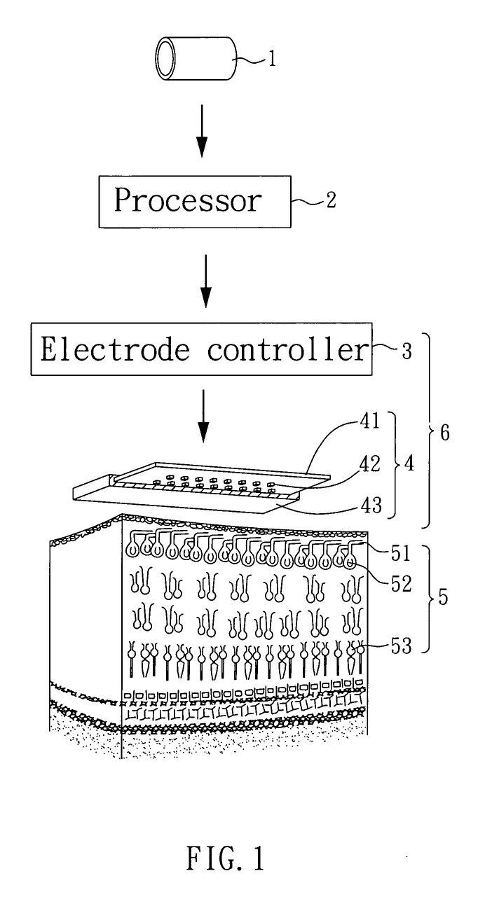

FIG. 1 is a perspective view of the electrical stimulation system for generating virtual channels of Embodiment 1 of the present invention, which is used as a retinal prosthesis. In the present embodiment, the electrical stimulation system is one kind of epiretinal prosthesis. First, the patient should wear glasses fitted with a micro-camera to receive the light images. Then, the light images received by the micro-camera are transferred into a control signal, which can be processed by the electrical stimulation system for generating virtual channels of the present embodiment, through a processor 2.

The electrical stimulation system for generating virtual channels of the present embodiment comprises: an electrode controller 3, a carrier 41, a plurality of electrode units 42, and a buffer layer 43. In the electrical stimulation system of the present embodiment, electrode units 42 are disposed on the carrier 41, and each of the electrode units 42 is electrically connected to the electrode controller 3 independently. Besides, the carrier 41 and the electrode units 42 are covered with the buffer layer 43. Additionally, the electrode units 42 are arranged in an array. In the present embodiment, the electrode units 42 are arranged in an 8×8 array. The electrode array 4 composed of the carrier 41, the electrode units 42, and the buffer layer 43 are disposed on the nerve fiber layer 51 of the retina 5, so the electrode array 4 of the present invention is one type of epiretinal prosthesis.

With reference to FIG. 1, the electrode controller 3 is electrically connected to the processor 2, so the control signal is transferred into the electrode controller 3 after processed by the processor 2. The electrode controller 3 drives the corresponding electrode units 42 after receiving the control signal, and the currents output from the corresponding electrode units 42 electrically interfere with each other in the buffer layer 43, to generate a virtual channel between the corresponding electrode units 42.

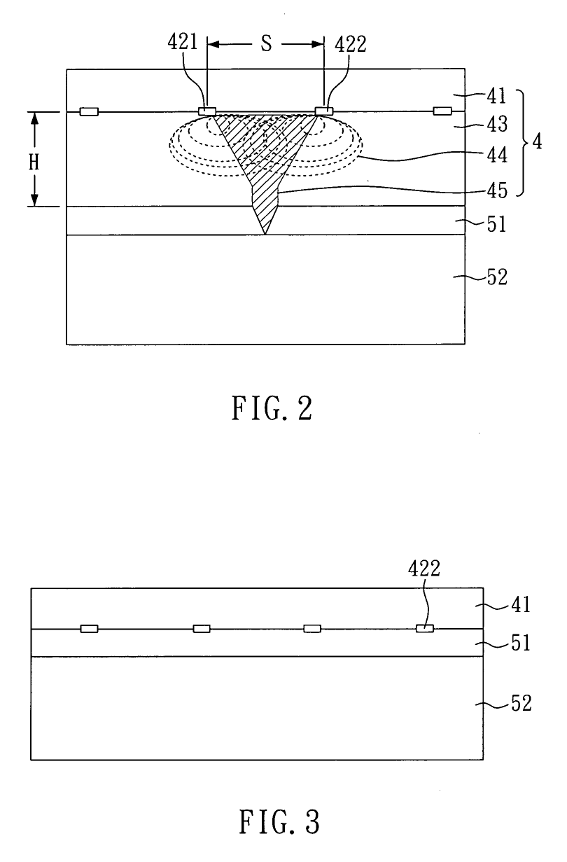

FIG. 2 is a cross-sectional view of the electrical stimulation system for generating virtual channels installed on the retinal in the present embodiment. The carrier 41 and the electrode units 421, 422 are covered with the buffer layer 43 to prevent the electrode units 421, 422 from contacting with the nerve fiber layer 51 directly. Hence, the buffer layer 43 can protect the nerve fiber layer 51 from being stimulated directly.

The electrical stimulation system for generating virtual channels of the present invention can be applied to medical devices and implants. Therefore, in the electrical stimulation system for generating virtual channels of the present invention, the material of the buffer layer 43 and the carrier 41 are biocompatible materials. Furthermore, the buffer layer 43 can further comprise a buffer liquid solution, wherein the components of the buffer solution are similar to the components of tissue fluid. In addition, the currents output from the electrode units 42 interfere with each other in the buffer layer 43 to generate a virtual channel, so the material of the buffer layer 43 has to be a semiconducting material, and the conductivity of the buffer layer is within a range of 0.01 to 100 simens/m. In the present embodiment, the conductivity of the buffer layer 43 is 1.43 simens/m. Also, the material of the carrier 41 has to be an insulating material, such as silicone, polyimide, or fluoropolymer resin. In the present embodiment, the material of the carrier is silicone.

The virtual channel is generated through the currents output from the electrode units interfering with each other, so the space for the generation of a virtual channel should be in a suitable range. If the distance between the electrode array and the nerve fiber layer is long enough, the virtual channel can be generated easily. However, as shown in FIG. 2, the electrode array 4 of the present invention is installed over the nerve fiber layer 51, so the space (between the electrode array 4 and the nerve fiber layer 51) for generating the virtual channel is restricted, due to the physiology structure of the retina. Hence, the thickness H of the buffer layer 43 is restricted. Preferably, the thickness H of the buffer layer 43 is within a range of 5 to 10000 μm. Moreover, the distance S between the electrode units 421, 422 is also restricted. If the distance S between the electrode units 421, 422 is too long, the currents output from the electrode units can not interfere with each other sufficiently and probably. Hence, the virtual channel cannot be generated when the distance S between the electrode units 421, 422 is not within a suitable range. Preferably, the distance between the electrode units 421, 422 may be within a range of 10 to 10000 μm. In the present embodiment, the distance S between the electrode units 421, 422 is within a range of 30 μm, and the thickness H of the buffer layer 43 is 15 μm.

With reference to FIGS. 1 and 2, the electrical stimulation method of the present invention comprises the following steps:

providing an electrical stimulation system 6, which comprises: an electrode controller 3, and an electrode array 4 (as shown in FIG. 1);

inputting a control signal to the electrode controller 3 of the electrical stimulation system 6, and driving the corresponding electrode units 421, 422 to output currents, which generate corresponding electric fields 44 (as shown in FIG. 2);

generating a virtual channel (or a stimulating site) 45 in the buffer layer 43, between the corresponding electrode units 421, 422, through the corresponding electric fields 44 generated from the currents output from the corresponding electrodes 421, 422 electrically interfering with each other.

With reference to FIG. 2, the corresponding electrode units driven by the electrode controller (not shown in FIG. 2) comprise at least two adjacent electrode units 421, 422. The virtual channel 45 is formed between two adjacent electrode units 421, 422, through the currents output from two adjacent electrode units 421, 422 interfering with each other. In addition, the position of the virtual channel 45 can be controlled by means of adjusting the current ratio output from the two adjacent electrode units 421, 422, wherein the current ratio can be adjusted by the electrode controller (not shown in FIG. 2).

Hence, the electrical stimulation system and method for generating virtual channels of the present embodiment not only can directly stimulate the nerve fiber corresponding to the positions of the electrode units, but also can stimulate the nerve fiber located outside the positions of the electrode units. Therefore, the electrical stimulation system and method for generating virtual channels of the present embodiment can improve the spatial resolution of the neural stimulation.

### Comparative Example 1

The electrical stimulation system of the present comparative example is similar that of the Embodiment 1, except that the electrical stimulation system of the present comparative example does not comprise a buffer layer. Hence, when the electrical stimulation system of the present comparative example is installed on the nerve fiber layer 51 of the retina, the electrode units 422 disposed on the carrier 411 are in contact with the nerve fiber layer 41, as shown in FIG. 3. Due to the close proximity of the electrode unit 422 to the nerve fiber layer 41, virtual channels can not be generated or adjusted.

Simulation Experiment Result

The activation of the nerve fiber of the retina excited by the electrical stimulation system of Embodiment 1 and Comparative example 1 is modeled with activation function (AF). The AF contour is numerical computed from the potential extracted from the modeling retina model. The AF contour presents the likelihood of exciting the nerve fiber, and the AF contour peak can be used to indicate the activated position, where the nerve fiber is most likely to be excited.

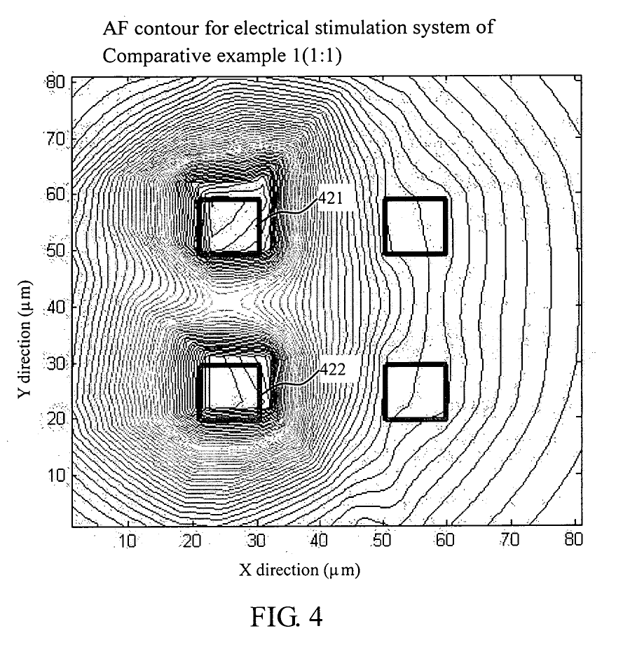

FIG. 4 is a stimulation model of the electrical stimulation system of Comparative example 1. When the voltage ratio input into electrode units 421, 422 is 1:1, there are two peaks under the electrode units 421, 422 and those indicate two excitations. From the modeling result show in FIG. 4, it is found that each of the electrode units 421, 422 generates stimulation to stimulate nerve fibers respectively.

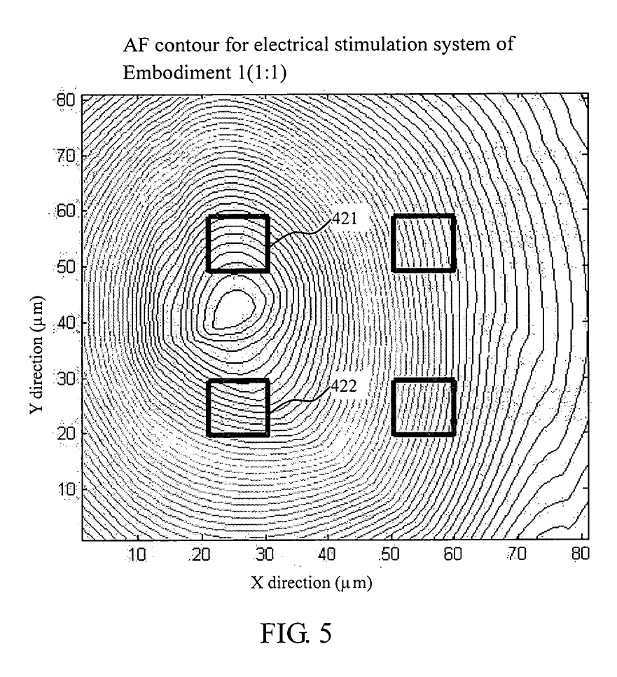

FIG. 5 is a stimulation model of the electrical stimulation system for generating virtual channels of Embodiment 1 of the present invention. When the voltage ratio input into electrode units 421, 422 is 1:1, there is only one peak between the two electrode units 421,422 and in the middle of the two electrode units 421, 422. Herein, the peak shown in FIG. 5 is the so-called virtual channel.

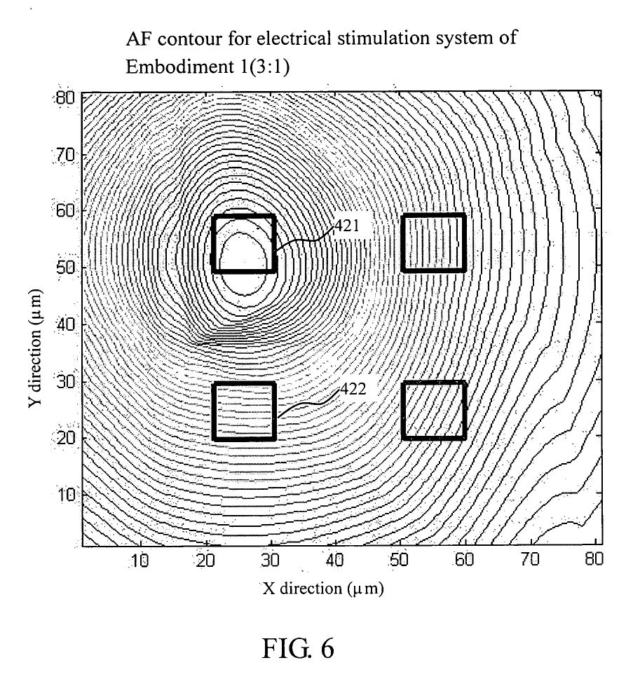

FIG. 6 is another stimulation model of the electrical stimulation system for generating virtual channels of Embodiment 1 of the present invention. When the voltage ratio input into electrode units 421, 422 is 3:1, there is only one peak between the two electrode units 421, 422, and that is the so-called virtual channel. Compared to the modeling result shown in FIG. 5, the position of the virtual channel shown in FIG. 6 is close to the electrode unit 421. From the modeling results shown in FIGS. 5 and 6, it is found that the position of the virtual channel can be controlled through adjusting the voltage (or current) input ratio into the electrode units, so it is possible to improve the spatial resolution of the neural stimulation.

From the modeling result shown in FIG. 4, the electrical stimulation system of Comparative example 1 can only stimulate the nerve fiber beneath the electrode units, because the electrical stimulation system of Comparative example does not comprise a buffer layer to generate the virtual channel. In contrast, from the modeling results shown in FIGS. 5 and 6, the electrical stimulation system of Embodiment 1 can generate virtual channels between the electrode units, because the electrical stimulation system of Embodiment 1 comprises a buffer layer. With a buffer layer, there is a space between the electrode units and nerve fibers. Therefore, the space allows the current output from the electrode units electrically to interfere with each other, and virtual channels can be generated more easily. In addition, the position of the virtual channels can be controlled through adjusting the voltage (or current) input ratio into the electrode units.

### Embodiment 2

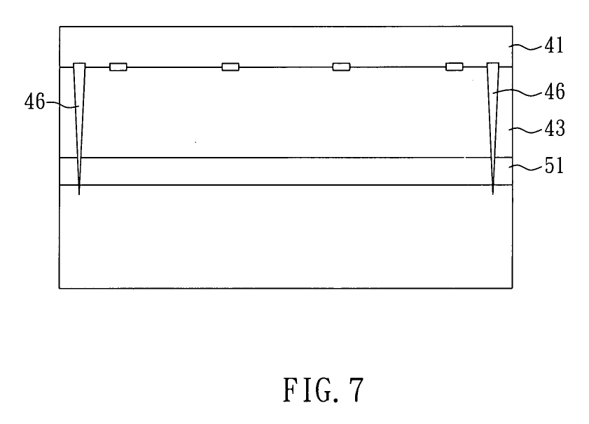

The electrical stimulation system for generating virtual channels of the present embodiment is similar to that of Embodiment 1, except that the electrical stimulation system of the present embodiment further comprises at least one anchor, which can be installed on the carrier or on the buffer layer to fix the electrode array on the nerve fiber layer of the retina. In the present embodiment, as shown in FIG. 7, the anchors 46 are installed on the carrier 41 and protrude from the outer surface of the buffer layer 43 to fix the electrode array (not shown in FIG. 7) on the nerve fiber layer 51 on the retina.

### Embodiment 3

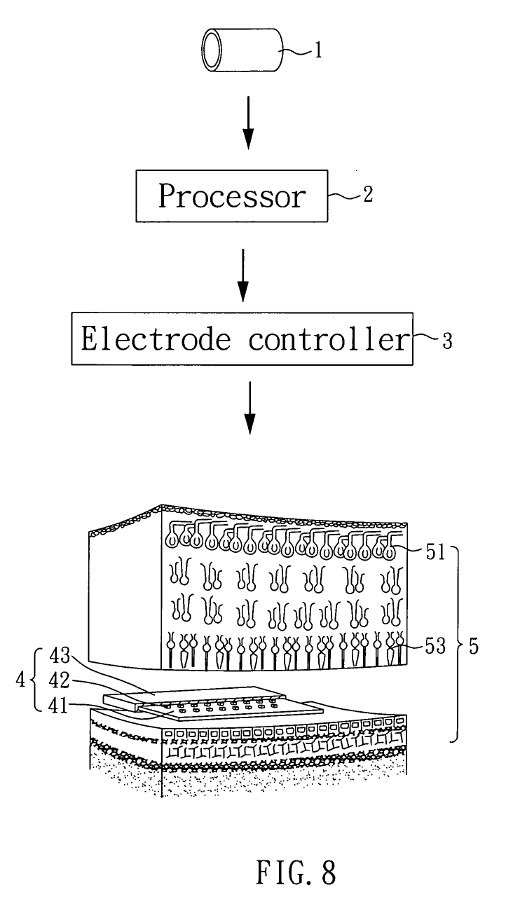

FIG. 8 is a perspective view of the electrical stimulation system for generating virtual channels of Embodiment 3 of the present invention, which is used as a retinal prosthesis. In the present embodiment, the electrical stimulation system is one kind of subretinal prosthesis. The electrical stimulation system and method for generating virtual channels are similar to those of Embodiment 1, except that the electrode array 4 is disposed under the photoreceptor layer 53, and the electrode array 4 faces the photoreceptor layer 53. When the electrode controller 3 drives the electrode units 42, the currents output from the electrode units 42 can stimulate the photoreceptor layer 53. Then, the signal is transmitted to the nerve fiber layer through cells in the photoreceptor layer 53.

### Embodiment 4

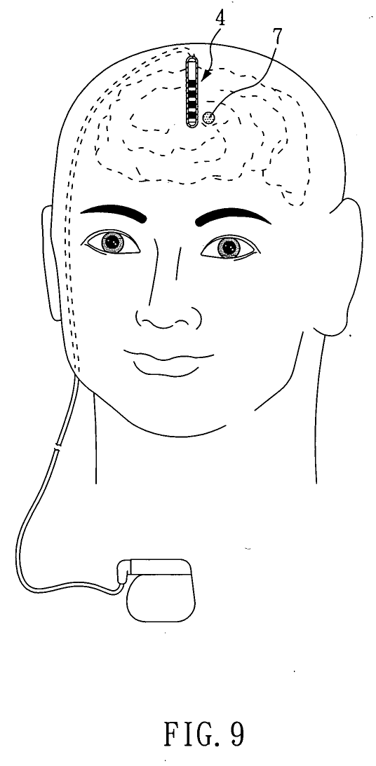

FIG. 9 is a perspective view of the electrical stimulation system for generating virtual channels used in the deep brain stimulation (DBS) in the present embodiment. The electrical stimulation system and method for generating virtual channels are similar to those of Embodiment 1, except that the electrode array 4 is a 1×4 array. The electrical stimulation system and method of the present embodiment can inhibit the abnormal brain excitation raised in the patient with brain disease, such as Parkinson's disease. Compared to the conventional electrical stimulation system used in DBS, the electrical stimulation system of the present embodiment can stimulate nerve fibers more accurately, due to the buffer layer. Hence, the electrical stimulation system of the present embodiment can further inhibit the abnormal excitation more exactly.

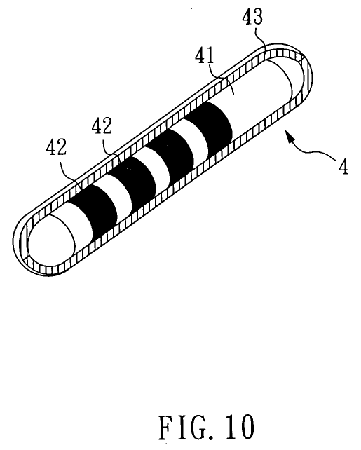

FIG. 10 is a perspective view of the electrical stimulation system for generating virtual channels in the present embodiment. With reference to FIGS. 9 and 10, the carrier 41 and the electrode units 42 of the present embodiment are covered with a buffer layer 43, so the position of the virtual channel generated from the electrode units 42 can be controlled through adjusting the current (or voltage) input ratio into the electrode units 42. Hence, the electrical stimulation system of the present embodiment can stimulate nerve fibers located between two adjacent electrode units 42, as shown in FIG. 9.

### Embodiment 5

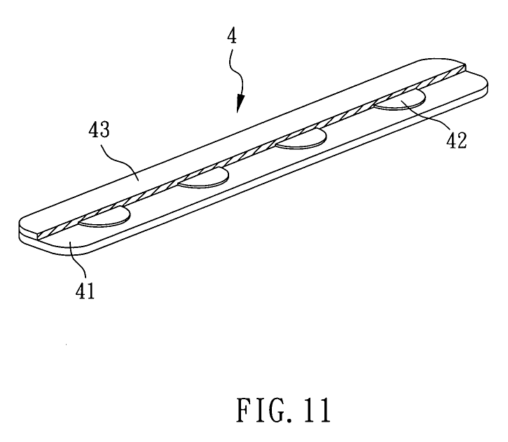

FIG. 11 is a perspective view of the electrical stimulation system for generating virtual channels used in the spinal cord stimulation (SCS) in the present embodiment. The electrical stimulation system and method for generating virtual channels are similar to those of Embodiment 1, except that the electrode array 4 is a 1×4 array.

The electrical stimulation system and method of the present embodiment can alleviate pain in patients with chronic back and/or leg pain. Compared to the conventional electrical stimulation system used in SCS, the electrical stimulation system of the present embodiment can stimulate nerve fibers located in the spinal cord more selectively, due to the buffer layer. Hence, the electrical stimulation system of the present embodiment can further alleviate chronic back and/or leg pain.

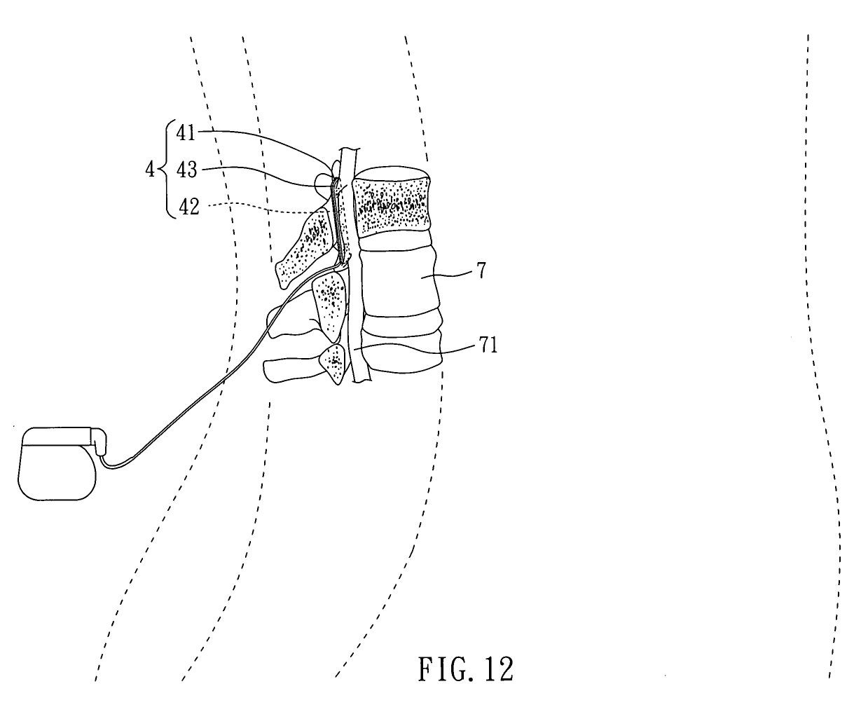

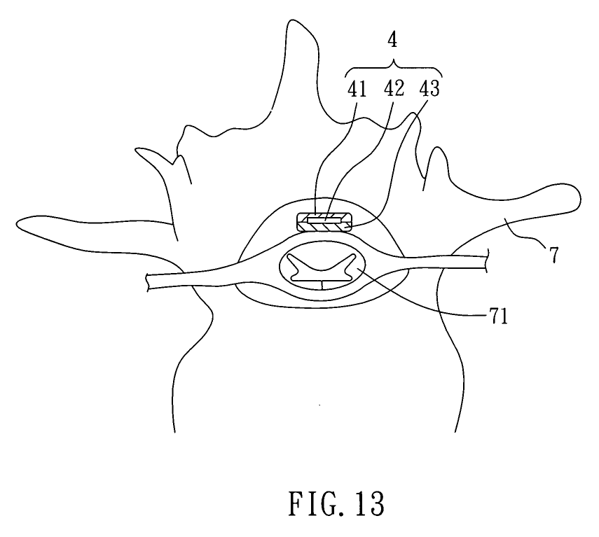

FIGS. 12 and 13 areis a perspective view and a cross-sectional view of the electrical stimulation system for generating virtual channels in the present embodiment, respectively. As shown in FIGS. 12 and 13, the electrical stimulation system 4 of the present embodiment is installed in the spinal column 7 to stimulate the nerve fibers located in the spinal cord 71. With reference to FIGS. 11, 12, and 13, the carrier 41 and the electrode units 42 of the present embodiment are covered with a buffer layer 43, so the position of the virtual channel generated from the electrode units 42 can be controlled through adjusting the current (or voltage) input ratio into the electrode units 42. Hence, the electrical stimulation system of the present embodiment can stimulate nerve fibers located inside the spinal cord 71 between two adjacent electrode units 42, as shown in FIG. 13.

In conclusion, the electrical stimulation system and method for generating virtual channels of the present invention can generate virtual channels between the two electrode units, because the buffer layer of the electrical stimulation system provides a space for virtual channel generation. However, it is hard to manufacture an electrode array with great quantity and high density of electrode units for electrical stimulation system, due to the limitation of the semiconductor process. In the conventional electrical stimulation system, it is unable to stimulate the nerve fibers between the electrode units under the limited number of electrode units, so the spatial resolution of the neural stimulation is not good enough. In contrast, when the electrical stimulation system and method for generating virtual channels of the present invention are used, it is possible to control the position of the virtual channels through adjusting the voltage (or current) input ratio into the electrode units. Hence, the virtual channels generated by the electrical stimulation system of the present invention can stimulate the nerve fibers between the electrode units, so it is able to improve the spatial resolution of the neural stimulation.

Although the present invention has been explained in relation to its preferred embodiment, it is to be understood that many other possible modifications and variations can be made without departing from the scope of the invention as hereinafter claimed.

What is claimed is:

1. An electrical stimulation system for generating virtual channels, comprising:
an electrode controller;
a carrier;
a plurality of electrode units disposed on the carrier, and each of the electrode units are electrically connected to the electrode controller independently; and
a buffer layer covering the carrier and the electrode units;
wherein, the electrode controller receives a control signal to drive the corresponding electrode units, and the currents output from the corresponding electrodes units electrically interfere with each other to generate a virtual channel between the corresponding electrode units.

2. The electrical stimulation system as claimed in claim 1, wherein the corresponding electrode units driven by the electrode controller comprise at least two adjacent electrode units.

3. The electrical stimulation system as claimed in claim 2, wherein the virtual channel is generated between the at least two adjacent electrode units.

4. The electrical stimulation system as claimed in claim 1, wherein the electrode units form an m×n array, and each m, and n is independently an integer of 1 or more.

5. The electrical stimulation system as claimed in claim 1, further comprising at least one anchor, which is disposed in the buffer layer and protrudes from the outer surface of the buffer layer.

6. The electrical stimulation system as claimed in claim 1, wherein the conductivity of the buffer layer is within a range of 0.01 to 100 simens/m.

7. The electrical stimulation system as claimed in claim 1, wherein the thickness of the buffer layer is within a range of 5 to 10000 μm.

8. The electrical stimulation system as claimed in claim 1, wherein the distance between the two adjacent electrode units is within a range of 10 to 10000 μm.

9. The electrical stimulation system as claimed in claim 1, wherein the material of the carrier is a biocompatible insulating material.

10. The electrical stimulation system as claimed in claim 1, wherein the material of the buffer layer is a biocompatible semiconducting material.

11. The electrical stimulation system as claimed in claim 1, wherein the electrical stimulation system is applied to retinal prosthesis, deep brain stimulation, or spinal cord stimulation.

12. An electrical stimulation method for generating virtual channels, comprising the following steps:
providing an electrical stimulation system, which comprises: an electrode controller, a carrier, a plurality of electrode units, and a buffer layer, wherein the electrode units are disposed on the carrier, each of the electrode units are electrically connected to the electrode controller independently, and the carrier and the electrode units are covered with the buffer layer;
inputting a control signal to the electrode controller of the electrical stimulation system, and driving the corresponding electrode units to output currents; and
generating a virtual channel between the corresponding electrode units through the currents output from the corresponding electrodes electrically interfering with each other.

13. The electrical stimulation method as claimed in claim 12, wherein the corresponding electrode units driven by the electrode controller comprise at least two adjacent electrode units.

14. The electrical stimulation method as claimed in claim 13, wherein the virtual channel is generated between the at least two adjacent electrode units.

15. The electrical stimulation method as claimed in claim 12, wherein the electrode units form an m×n array, and each m, and n is independently an integer of 1 or more.

16. The electrical stimulation method as claimed in claim 12, wherein the electrical stimulation system further comprises at least one anchor, which is disposed in the buffer layer and protrudes from the outer surface of the buffer layer.

17. The electrical stimulation method as claimed in claim 12, wherein the conductivity of the buffer layer is within a range of 0.01 to 100 simens/m.

18. The electrical stimulation method as claimed in claim 12, wherein the thickness of the buffer layer is within a range of 5 to 10000 μm.

19. The electrical stimulation method as claimed in claim 12, wherein the distance between the two adjacent electrode units is within a range of 10 to 10000 μm.

20. The electrical stimulation method as claimed in claim 12, wherein the material of the carrier is a biocompatible insulating material.

21. The electrical stimulation method as claimed in claim 12, wherein the material of the buffer layer is a biocompatible semiconducting material.

22. The electrical stimulation method as claimed in claim 12, wherein the electrical stimulation method is applied to retinal prosthesis, cochlear prosthesis, deep brain stimulation, or spinal cord stimulation
